(19) United States
(12) Patent Application Publication
(10) Pub. No.: US 2025/0177046 A1
(43) Pub. Date: Jun. 5, 2025

(54) METHOD, COMPUTING DEVICE, SYSTEM AND COMPUTER PROGRAM PRODUCT TO SUPPORT SELECTION BETWEEN DIFFERENT TYPES OF PEDICLE SCREWS TO BE APPLIED IN A SPINE OF A SPECIFIC PATIENT

(71) Applicant: 25SEGMENTS AG, CH-8008 ZÜRICH (CH)

(72) Inventors: Jonas WIDMER, Zürich (CH); Marie-Rosa FASSER, Zürich (CH); Mazda FARSHAD, Zumikon (CH)

(73) Assignee: 25SEGMENTS AG, CH-8008 ZÜRICH (CH)

(21) Appl. No.: 18/845,041

(22) PCT Filed: Mar. 7, 2023
(86) PCT No.: PCT/EP2023/055648

§ 371 Date: Sep. 9, 2024

(30) Foreign Application Priority Data
Mar. 8, 2022 (CH) CH000245/2022

(51) Int. Cl. A61B 34/10

(52) U.S. Cl. CPC A61B 34/10; A61B 2034/102; A61B 2034/105; A61B 2034/108

(57) ABSTRACT

Computer implemented method, computing device, system and computer pro-gram product to support selection between different types of pedicle screws (20₁₋ₘ) to be applied in a spine (202) comprising: generating a spinal 3D-model (M_{Spine}) of a patient; generating a reinforcement 3D-model (M_{Rod}) of a reinforce-5 ment structure (30) for interconnecting vertebrae (200₁₋ₙ) of the spine (202); generating a finite element model (FE) corresponding to the spinal 3D-model (M_{Spine}) and the reinforcement 3D-model (M_{Rod}); applying a load onto the finite element model (FE) and determining loading factor(s) of the pedicle screws (20₁₋ₘ) using the finite element model (FE); and outputting an assessment of the plurality of different types of pedicle screws (20₁₋ₘ) based on the calculated loading factor(s).

### BACKGROUND OF THE INVENTION

### Field of the Invention

The present invention relates to a computer implemented method, computing device, system and computer program product to support selection between different types of pedicle screws to be applied in a spine of a specific patient.

### Discussion of Related Art

Posterior spinal fixation of the lower spine is a gold standard surgical intervention and is employed for spine stabilization through the fusion of spinal segments. Pedicle screw loosening after spondylodesis surgery is a common complication that can lead to persistent pain, failed fusion, and the need for revision surgery. Screw loosening has been reported to occur in about 10% of patients, and this percentage increases to above 60% for patients affected by osteoporosis. Hence there is a need to predict the risk of screw loosening through the examination of subject-specific biomechanical aspects for the improvement of surgical outcomes.

There is no prior art quantitative and reliable measure to identify level-specific risk of loosening besides a general evaluation of bone quality from image data.

Finite element and musculoskeletal modeling are known methods in biomechanical research and can be complementary to each other. However, until now, the clinical relevance and impact of such models have been hampered by the circumstance that the gained insight could usually not be directly translated into improved patient care.

### SUMMARY OF THE INVENTION

It is an object of the present invention to provide a computer implemented method, computing device, system and computer program product to support selection between different types of pedicle screws to be applied in a spine of a specific patient that overcomes one or more of the disadvantages of the prior art.

According to the present disclosure, this object is addressed by the features of the independent claims. In addition, further advantageous embodiments follow from the dependent claims and the description.

In particular, it is an object of the present invention to provide a computer implemented method to support selection between different types of pedicle screws to be applied in a spine of a specific patient which allow identifying a pedicle screw type minimizing the risk of pedicle screw loosening from the spine of the specific patient.

In the context of the present specification, the term “different types of pedicle screws” refers to pedicle screws having different geometries (such as length, diameter), materials, or different fixation means. According to particular embodiments, the present invention supports the selection between traditional, expandable or cement-augmented pedicle screws.

In particular, the above object is addressed according to the present invention by a computer implemented method to support selection between different types of pedicle screws to be applied in a spine of a specific patient comprising the following method steps carried out by a computing device:
a. generating a spinal 3D-model of at least part of a spine;
b. generating a 3D-model of a reinforcement structure;
c. generating a finite element model corresponding to the spinal 3D-model and the reinforcement 3D-model;
d. applying load onto the finite element model and determining a loading factor of at least one of the pedicle screws; and
e. outputting an assessment of the plurality of different types of pedicle screws based on the calculated loading factor.

Inventors of the present invention recognized that known finite element-based methods of assessing the risk of loosening of pedicle screws failed to translate into improved patient care at least for the reason that known finite element-based methods, of assessing the risk of loosening of pedicle screws, are limited to the assessment of a pull-out force of individual pedicle screws.

The present invention addresses this shortcoming of known finite element-based methods at least in that the finite element model of the present invention is constructed such as to represent the pedicle screws as part of a reinforcement structure attached to the vertebrae using the pedicle screws, thereby enabling a holistic analysis of the entire reinforcing structure in order to facilitate selection of the appropriate pedicle screws. In other words, while prior art methods focus on providing local optima for individual pedicle screws, the present invention aims to provide a global optimum based on a finite element model-based analysis of the entire reinforcing structure as attached to the plurality of vertebrae using the corresponding pedicle screws.

### Generating a Spinal 3D-Model

The computing device is configured to generate a spinal 3D-model of at least part of a spine of the specific patient. The spinal 3D-model captures at least two vertebrae of the patient's spine.

According to embodiments disclosed herein, the spinal 3D-model is generated using a database of 3D-models of spines, in particular a database comprising information of 3D-models of spines of people other than the specific patient.

Alternatively, or additionally, the spinal 3D-model is generated based on preoperative image(s), such as radioscopic images, e.g. as Computed Tomography (CT) image(s) of the patient; low-dose Computed Tomography (CT) and/or MRI based pseudo-Computed Tomography images.

In particular embodiments, the spinal 3D-model is generated based on a CT image(s) of the patient by an artificial intelligence-based model, the artificial intelligence-based model having been trained using a dataset of CT images (of people other than the patient) along with corresponding vertebral level annotations.

According to embodiments disclosed herein, if present in the patient's spine, intervertebral disc(s) and/or fusion cage(s) arranged between the at least two vertebrae are also captured by the spinal 3D-model.

### Generating a Reinforcement 3D-Model

The computing device is further configured to generate a reinforcement 3D-model of a reinforcement structure for interconnecting at least two vertebrae of the spine.

Commonly, the reinforcement structure comprises a reinforcement rod for being attached to at least two pedicle screws to thereby interconnect the respective vertebrae, in particular by connecting to U-shaped openings of the pedicle screw heads. The reinforcement structure is specific to the positioning of the pedicle screws within the patient's spine, being shaped according to the spatial positions of a plurality of chirurgical implants (pedicle screws) attached to a patient.

The locations of the pedicle screws, to be attached to the vertebrae, are determined based on the spinal 3D-model and are specific to the patient.

According to embodiments disclosed herein, the locations of the pedicle screws are determined using an artificial intelligence-based model, supervised-learning and/or reinforcement learning being used to train the artificial intelligence-based model using a dataset comprising expert-identified ideal screw trajectories.

Alternatively, or additionally, the locations of the pedicle screws are determined semi-automatically or manually by a user based on the spinal 3D-model. According to an embodiment, the locations of the pedicle screws are indicated by a user using a pointing device, the pointing device being tracked by a tracking device and translated into data indicative of the locations of the pedicle screws within the reinforcement 3D-model.

Thereafter, on the basis of the locations of the pedicle screws, the reinforcement 3D-model is further generated such that the reinforcement structure is shaped to attach to at least two pedicle screws to thereby interconnect the respective vertebrae. The reinforcement structure is determined such as to form an alternative load path outside the interconnected vertebrae to thereby relieve at least part of the load from the respective vertebrae.

According to further embodiments, a spinal alignment process is applied onto the patient's spine before the reinforcement 3D-model is generated. The spinal alignment process is applied such that the anatomy of the spine is (at least partially) corrected.

Hence, the reinforcement 3D-model is generated such that, when the corresponding reinforcement structure is secured to the respective vertebrae using the pedicle screws, the posture of the patient is (at least partially) aligned, i.e. corrected according to an ideal/improved posture defined by a surgeon or orthopedic physician.

Finally, the reinforcement 3D-model comprises a digital representation of a reinforcement structure attached to the at least two pedicle screws.

### Generating a Finite Element Model

Once the spinal 3D-model and the reinforcement 3D-model are generated, a finite element model is generated corresponding to the spinal 3D-model and the reinforcement 3D-model.

The finite element model represents at least the following:
i. the at least two vertebrae as captured by the spinal 3D-model; and
ii. the reinforcement structure attached to the vertebrae using at least one pedicle screw per interconnected vertebrae.

According to embodiments disclosed herein, material properties indicative of a bone quality of the vertebrae are extracted from preoperative image(s) of the spine of the specific patient, in particular from preoperative CT images of the spine. The extracted material properties are then assigned to volume elements of the finite element model. Correspondingly, the loading factor of the at least one of the pedicle screws is determined as a relationship between a locally predicted stress and the bone quality of the corresponding vertebrae. In other words, the loading factor is indicative of the relationship between a predicted stress on the tissue and its resistance to failure.

The material properties comprise one or more of: stiffness; bone density and/or bone strength. According to an embodiment, bone density of the vertebrae is calculated based on the Hounsfield units HU determined from the preoperative CT images of the spine.

The pedicle screws modeled/represented in the finite element model are of a first type from the different types of pedicle screws, the support of a selection between these different types of pedicle screws being the object of the present invention.

According to further embodiments disclosed herein, pedicle screws of a second type, from the different types of pedicle screws, are also modeled using the finite element model, the output (i.e. the loading factors) of the finite element model being (re) calculated for the second type of pedicle screws. According to particular embodiments, the finite element model is generated for traditional, expandable and/or cement-augmented pedicle screws.

A screw type model is provided with respect to each type of pedicle screw represented by the finite element model. In particular, the screw type model comprises a 3D-surface model of the pedicle screw.

Applying Load onto the Finite Element Model

A load is then applied onto the finite element model to thereby determine a loading factor of at least one of the pedicle screws. In particular, the loading factor is determined as a relationship between a predicted stress on the vertebrae and the vertebra's resistance to failure.

Inventors of the present invention further recognized that known finite element-based methods of assessing the risk of loosening of pedicle screws failed to translate into improved patient care at least for the reason that an axial pull-out strength(s) of the pedicle screws, on which known finite element-based methods are based, had reduced correlation with pedicle screw loosening in patients.

Further embodiments of the present invention address this shortcoming by applying the load onto the finite element model (representing the pedicle screws as part of the entire reinforcement structure) in a caudo-cranial direction. In other words, the load is applied as if pulling the vertebra down with respect to the pedicle screws. According to further embodiments, a patient-specific caudo-cranial direction is determined based on the spinal 3D-model of the specific patient.

According to embodiments disclosed herein, load of the finite element model is applied onto the pedicle screw attached to the uppermost vertebrae (of the plurality of vertebrae interconnected by the reinforcement structure).

### Outputting an Assessment of Different Types of Pedicle Screws

An assessment of the different types of pedicle screws is generated based on the loading factor(s) determined using the finite element model generated for at least a first type of pedicle screws. According to embodiments disclosed herein, the assessment of the plurality of different types of pedicle screws comprises comparing the load factor of one or more pedicle screw(s) with a critical load factor threshold. A warning signal is generated with respect to each pedicle screw with a load factor exceeding a critical load factor threshold.

In embodiments where the loading factor is calculated, using the finite element model, only for the first type of pedicle screws, a selection between the different types of pedicle screws is supported by the assessment of the suitability of the first type of pedicle screws for the spine of the specific patient. In particular, the suitability of a pedicle screw is assessed by a comparison of its calculated load factor with a critical load factor threshold. The load factor of a pedicle screw of the first type exceeding the critical load factor threshold is indicative of the first type of pedicle screws not being suitable for the spine of the specific patient.

In order to even better support the selection between a plurality of types of pedicle screws, according to embodiments disclosed herein, the output of the finite element model is recalculated based on at least one pedicle screw of a second type of the different types of pedicle screws. The assessment may in such embodiments comprise a ranking of the different types of pedicle screws by their calculated loading factors. Whether the output of the finite element model needs to be recalculated for pedicle screws of a second or further type may be determined based on a comparison of the load factor (of the latest pedicle screw type analyzed) with the critical load factor threshold, the process being terminated upon identifying of a type of pedicle screws the load factor of which does not exceed the critical load factor threshold.

According to even further embodiments, the output of the finite element model is recalculated based on at least one pedicle screw positioned differently in the finite element model. In particular, if no type of pedicle screws could be identified with a load factor not exceeding the critical load factor threshold, the positioning of the pedicle screws may be adjusted and the finite element model regenerated in order to support a selection of pedicle screw type suitable considering the adjusted reinforcement 3D-model.

The assessment is then output to support selection between different types of pedicle screws to be applied in the spine of the specific patient. According to embodiments disclosed herein, the assessment is output by means of a display device communicatively connected to the computing device. Alternatively, or additionally, the assessment is output by the computing device as a data signal indicative of the assessment.

In order to achieve an even further accurate modelling of the spine of the specific patient, according to further embodiments, at least a partial patient specific musculoskeletal model is created based on individual anatomy, alignment, and mass distribution. For example, preoperative images of the patient, in particular annotated radiographs of the erect posture can be used for the generation of individualized musculoskeletal model. The individualized musculoskeletal model is used to determine a physiological load acting on the spine, in particular on joints of the spine, during standing. The joint reaction forces, resulting from the physiological load acting on the spine, are applied as load on the finite element model.

It is a further object of the present invention to provide a computing device to support selection between different types of pedicle screws to be applied in a spine of a specific patient which allow identifying a pedicle screw type minimizing the risk of pedicle screw loosening from the spine of the specific patient.

According to the present disclosure, this object is addressed by the features of the independent claims. In addition, further advantageous embodiments follow from the dependent claims and the description.

In particular, the above-identified object is further achieved by a computing device comprising a processing unit and a memory unit comprising instructions, which, when executed by the processing unit, cause the computing device to carry out the method according to one of the embodiment disclosed herein. The processing unit comprises any one or a combination of: a central processing unit CPU, a remote computing service, such as a cloud based software as a service, and/or a dedicated hardware circuitry, such as a ASIC or FPGA. The memory unit comprises any one or a combination of: a volatile or non-volatile, optic, magnetic or semiconductor based data storage device, and/or a cloud based datastore.

According to embodiments disclosed herein, the computing device comprises a data input interface communicatively connectable to an imaging device for capturing preoperative images of the patient. The data input interface, such as a wired (e.g. Ethernet, DVI, HDMI, VGA) and/or wireless data communication interface (e.g. 4G, 5G, Wifi, Bluetooth, UWB) is communicatively connectable to receive preoperative imaging data therefrom. The data output interface such as a wired (e.g. Ethernet, DVI, HDMI, VGA) and/or wireless data communication interface (e.g. 4G, 5G, Wifi, Bluetooth, UWB) is configured to transmit at least part of assessment of the plurality of different types of pedicle screws, respectively a visual representation of the assessment.

It is a further object of the present invention to provide a system to support selection between different types of pedicle screws to be applied in a spine of a specific patient which allow identifying a pedicle screw type minimizing the risk of pedicle screw loosening from the spine of the specific patient.

According to the present disclosure, this object is addressed by the features of the independent claims. In addition, further advantageous embodiments follow from the dependent claims and the description.

In particular, the above-identified object is further achieved by a system comprising an imaging device and a computing device according to one of the embodiments disclosed herein. According to a particular embodiment, the imaging device is a CT imaging device configured to capture preoperative CT image(s) of at least part of the spine capturing at least two vertebrae of the spine.

According to further embodiments, the system further comprises an output device, such as a computer screen, an augmented reality headset, or any device suitable to display a visual representation of the assessment of the plurality of different types of pedicle screws, the output device being communicatively connected to the computing device.

It is a further object of the present invention to provide a computer program product to support selection between different types of pedicle screws to be applied in a spine of a specific patient which allow identifying a pedicle screw type minimizing the risk of pedicle screw loosening from the spine of the specific patient.

According to the present disclosure, this object is addressed by the features of the independent claims. In addition, further advantageous embodiments follow from the dependent claims and the description.

In particular, the above-identified object is further achieved by a computer program product, comprising instructions, which, when carried out by a processing unit of a computing device, cause the computing device to carry out the method according to any one of the embodiments disclosed herein.

It is to be understood that both the foregoing general description and the following detailed description present embodiments, and are intended to provide an overview or framework for understanding the nature and character of the disclosure. The accompanying drawings are included to provide a further understanding, and are incorporated into and constitute a part of this specification. The drawings illustrate various embodiments, and together with the description serve to explain the principles and operation of the concepts disclosed.

### BRIEF DESCRIPTION OF SEVERAL VIEWS OF THE DRAWINGS

The herein described invention will be more fully understood from the detailed description given herein below and the accompanying drawings which should not be considered limiting to the invention described in the appended claims.

FIG. 1 shows a flowchart illustrating the steps according to a particular embodiment of the method to support selection between different types of pedicle screws;

FIG. 2 shows a highly schematic perspective view of visual representations of a spinal 3D-model and of a reinforcement 3D-model of a reinforcement structure;

FIG. 3 shows an illustration of extracting material properties indicative of a bone quality of the vertebrae from preoperative CT images of the spine;

FIG. 4 shows a sequence of illustrations of applying a spinal alignment onto a patient's spine, generating a spinal 3D-model, generating a musculoskeletal model respectively of a finite element model representing the musculoskeletal model as well as the reinforcement structure;

FIG. 5 shows a visual representation of a finite element model according to the present invention, illustrating the load applied onto the finite element model in a caudo-cranial direction;

FIG. 6 shows a partial visual representation of a finite element model according to the present invention, illustrating the load on a single pedicle screw;

FIG. 7 shows a visual representation of a finite element model according to the prior art, for calculating a pull-out strength of a single pedicle screw for assessing a pedicle screw, the load being applied in an axial direction (of the pedicle screw);

FIG. 8 shows a partial visual representation of a finite element model according to the present invention, illustrating applying the load onto the finite element model in a caudo-cranial load;

FIG. 9 shows a visual representation of a finite element model according to the present invention, illustrating the local predicted stress in the bone structure of a pedicle around a pedicle screw; and

FIG. 10 shows a highly schematic block diagram of a system for supporting selection between different types of pedicle screws according to the present invention.

### DETAILED DESCRIPTION OF THE INVENTION

Reference will now be made in detail to certain embodiments, examples of which are illustrated in the accompanying drawings, in which some, but not all features are shown. Indeed, embodiments disclosed herein may be embodied in many different forms and should not be construed as limited to the embodiments set forth herein; rather, these embodiments are provided so that this disclosure will satisfy applicable legal requirements. Whenever possible, like reference numbers will be used to refer to like components or parts.

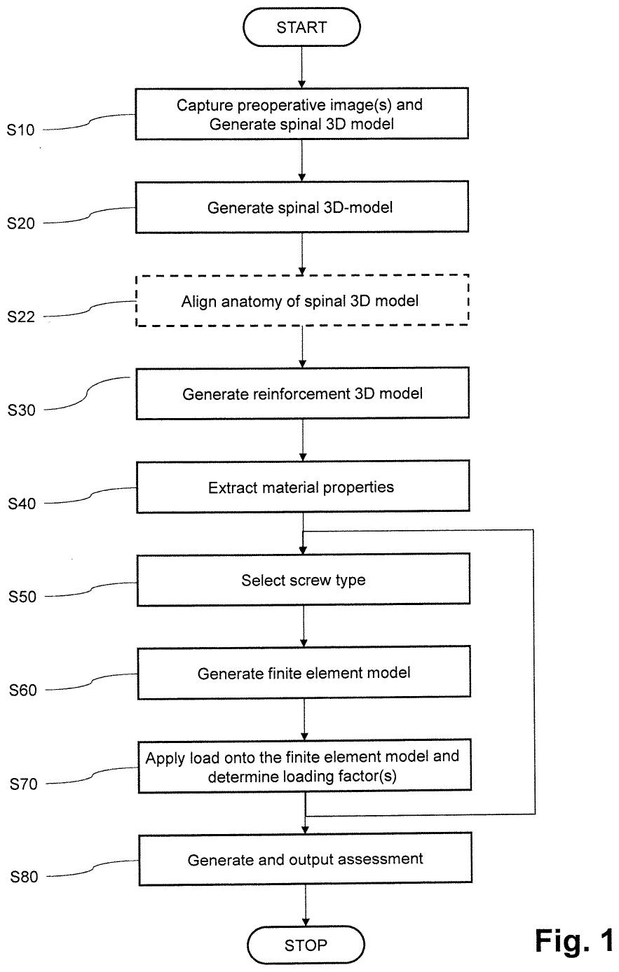

FIG. 1 shows a flowchart illustrating the steps according to a particular embodiment of the method to support selection between different types of pedicle screws 20₁₋ₘ.

In a preparatory step S10, radioscopic preoperative image(s), such as CT image(s), of the patient are captured using an imaging device 50, such as a CT imaging device, of at least part of the spine 202 and at least two vertebrae 200₁₋ₙ of the spine 202.

Based on the preoperative images, in step S20, the computing device 10 generates a spinal 3D-model M_{Spine} of at least part of the spine 202 of the specific patient, capturing at least two vertebrae 200₁₋ₙ of the patient's spine 202. If present in the patient's spine, intervertebral disc(s) and/or fusion cage(s) arranged between the at least two vertebrae 200₁₋ₙ. are also captured by the spinal 3D-model M_{Spine}. The spinal 3D-model M_{Spine} is generated based on CT image(s) of the patient by an artificial intelligence-based model, the artificial intelligence-based model having been trained using a dataset of CT images (of people other than the patient) along with corresponding vertebral level annotations.

Thereafter, in optional step S22, a spinal alignment process is applied onto the patient's spine before the reinforcement 3D-model M_{Rod} is generated. The spinal alignment process is applied such that the anatomy of the spine is (at least partially) corrected-when the reinforcing structure is implanted.

In subsequent step S30, the computing device 10 generates a reinforcement 3D-model M_{Rod} of a reinforcement structure 30 for interconnecting at least two vertebrae 200₁₋ₙ of the spine 202. The reinforcement structure 30 comprises a reinforcement rod for being attached to pedicle screws 20₁₋ₘ to thereby interconnect the respective vertebrae 200₁₋ₙ, by connecting to U-shaped openings of the pedicle screws 20₁₋ₘ. The reinforcement structure 30 is specific to the positioning of the pedicle screws 20₁₋ₘ within the patient's spine 202, being shaped according to the spatial positions of a plurality of chirurgical implants 20₁₋ₘ (pedicle screws) attached to a patient.

The locations of the pedicle screws 20₁₋ₘ to be attached to the vertebrae 200₁₋ₙ—are determined based on the spinal 3D-model and are specific to the patient. The reinforcement structure 30 forms an alternative load path outside the interconnected vertebrae 200₁₋ₙ to thereby relieve at least part of the load from the respective vertebrae 200₁₋ₙ.

If an alignment process has been applied to (at least partially) correct the anatomy of the spine 202, the reinforcement 3D-model M_{Rod} is generated such that, when the corresponding reinforcement structure 30 is secured to the respective vertebrae 200₁₋ₙ using the pedicle screws 20₁₋ₘ, the posture of the patient is (at least partially) aligned, i.e. corrected according to an ideal/improved posture defined by a surgeon or orthopedic physician.

In step S40, material properties indicative of a bone quality of the vertebrae 200₁₋ₙ are extracted from preoperative image(s) of the spine 202 of the specific patient, in particular from preoperative CT images of the spine 202.

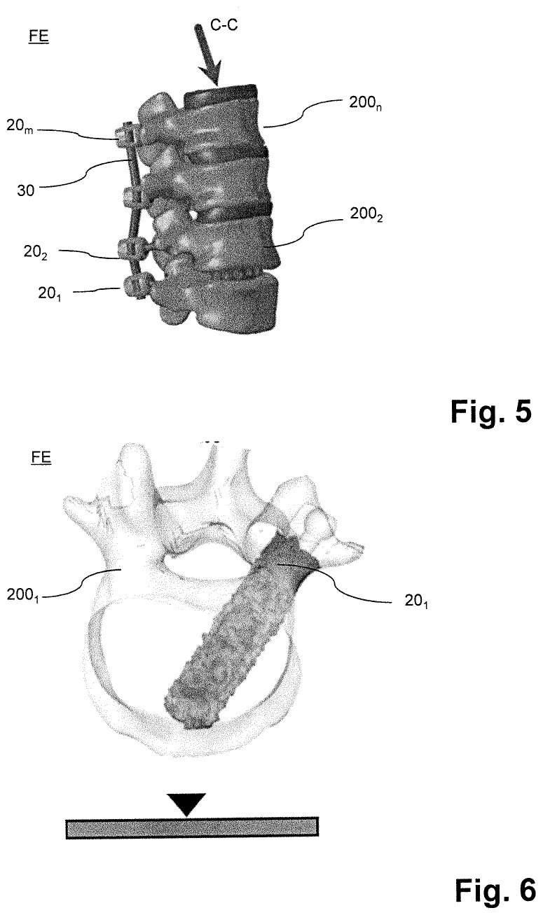

In the sequence of steps S50, S60 and S70, a loading factor (of at least one of the pedicle screws 20₁₋ₘ) is repeatedly calculated for each type of pedicle screw to be assessed. In step S50, a specific type of pedicle screw is selected from a plurality of different types of pedicle screws (such as traditional, expandable or cement-augmented pedicle screws). Thereafter, having selected a specific type of pedicle screw, in step S60, a finite element model FE is generated corresponding to the spinal 3D-model M_{Spine} and the reinforcement 3D-model M_{Rod}. The extracted material properties are assigned to volume elements of the finite element model FE. The generation of the finite element model FE is described in detail with reference to FIG. 5. In step S70, a caudo-cranial load is then applied onto the finite element model FE, for example on the uppermost vertebra 200n and a loading factor is calculated for at least one of the pedicle screws 20₁₋ₘ. Calculation of the loading factor is described in detail with reference to FIG. 5. The loading factor is determined as a relationship between a locally predicted stress and the bone quality of the corresponding vertebrae 200₁₋ₙ.

The sequence of steps S50, S60 and S70 is repeated until a loading factor has been determined for all pedicle screw types to be assessed. Thereafter, in step S80, an assessment of the different types of pedicle screws 20₁₋ₘ is generated based on the loading factor(s) determined using the finite element models FE. The assessment of the plurality of different types of pedicle screws 20₁₋ₘ comprises comparing the load factor of one or more pedicle screw(s) 20₁₋ₘ with a critical load factor threshold. A warning signal is generated with respect to each pedicle screw 20₁₋ₘ with a load factor exceeding a critical load factor threshold. Alternatively, or additionally, an assessment table is output comprising a listing of the loading factors of each of the assessed pedicle screw type. Optionally, a recommendation is generated for selecting a screw type based on the loading factors of each of the assessed pedicle screw type.

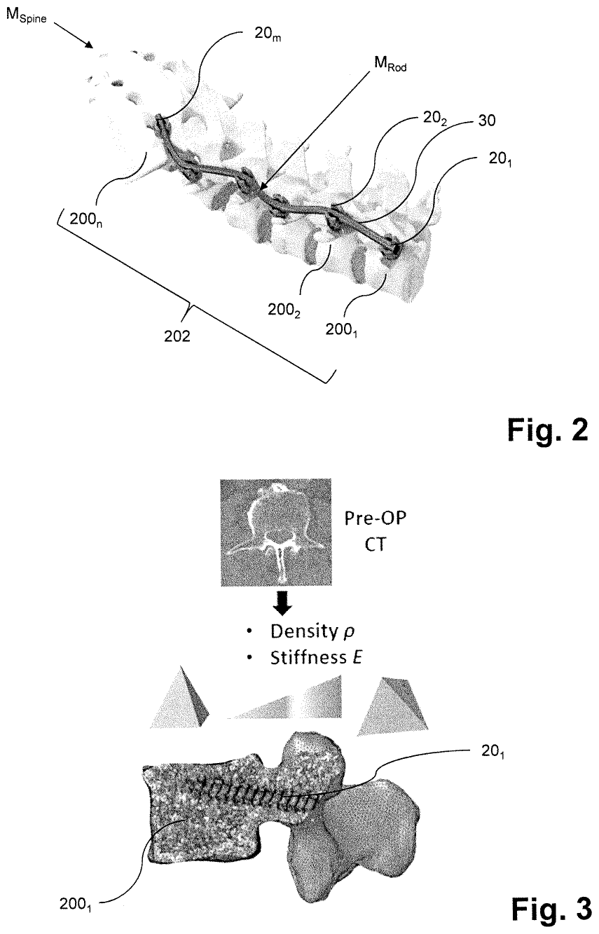

FIG. 2 shows a highly schematic perspective view of a visual representation of a spinal 3D-model M_{Spine} (shown in light grey) and of a reinforcement 3D-model M_{Rod} of a reinforcement structure 30 (shown in dark grey). The spinal 3D-model M_{Spine} captures multiple vertebrae 200₁₋ₙ of the patient's spine 202. As illustrated, intervertebral disc(s) are also captured by the spinal 3D-model M_{Spine}. Overlaid onto the spinal 3D-model M_{Spine}, FIG. 2 also depicts the reinforcement 3D-model M_{Rod} of a reinforcement structure 30 for interconnecting multiple vertebrae 200₁₋ₙ of the spine 202. In the illustrated embodiments, the reinforcement structure 30 is a reinforcement rod for being attached to U-shaped openings (so-called tulips) of the pedicle screw heads. The reinforcement structure 30 is specific to the positioning of the pedicle screws within the patient's spine, being shaped according to the spatial positions of a plurality of chirurgical implants (pedicle screws) attached to a patient.

FIG. 3 illustrates how material properties indicative of a bone quality of the vertebrae 200₁₋ₙ, such as density and stiffness, are extracted from preoperative CT images of the spine 202. Bone density of the vertebrae is calculated based on the Hounsfield units HU determined from the preoperative CT images of the spine 202. In order to improve accuracy, variable tube voltage used for acquisition of the preoperative CT images may be accounted for by correcting the HU values.

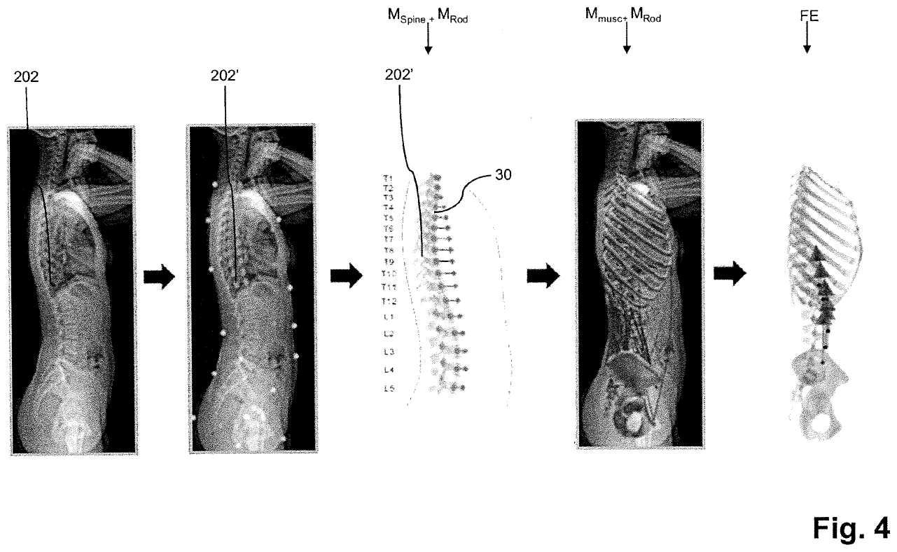

FIG. 4 depicts a sequence of illustrations of applying a spinal alignment onto a patient's spine 202, generating a spinal 3D-model M_{Spine}, generating a musculoskeletal model M_{Muse}, respectively of a finite element FE model representing the musculoskeletal model M_{Muse} as well as the reinforcement structure 30.

The leftmost illustration of FIG. 4 depicts a preoperative radiographic image of a patient's spine 202 as captured by an imaging device 50. The second illustration from the left of FIG. 4 depicts an aligned spine 202′ after a spinal alignment process has been applied onto the spine 202. The spinal alignment process is applied such that the anatomy of the spine is (at least partially) corrected. The illustration in the middle of FIG. 4 depicts a visual representation of the spinal 3D-model M_{Spine}, generated after the spinal alignment overlaid onto a visual representation of the reinforcement 3D-model M_{Rod} generated on the basis of the spinal 3D-model M_{Spine}. The second illustration from the right on FIG. 4 depicts a visual representation of a musculoskeletal model M_{Muse} generated based on individual anatomy, alignment, and mass distribution as determined based on preoperative images of the patient.

Finally, the rightmost illustration of FIG. 4 depicts a visual representation of the finite element model FE representing the musculoskeletal model M_{Muse}, the multiple vertebrae 200₁₋ₙ as captured by the spinal 3D-model M_{Spine} and further representing the reinforcement structure 30 attached to the vertebrae 200₁₋ₙ using pedicle screws 20₁₋ₘ.

FIG. 5 shows a visual representation of the finite element model FE according to the present invention, illustrating the load applied onto the finite element model in a caudo-cranial direction C-C. The finite element model FE is generated corresponding to the spinal 3D-model M_{Spine} and the reinforcement 3D-model M_{Rod}. Therefore, the finite element model FE represents the vertebrae 200₁₋ₙ as captured by the spinal 3D-model M_{Spine}; and the reinforcement structure 30 attached to the vertebrae 200₁₋ₙ using at least the pedicle screws 20₁₋ₘ.

As illustrated with a thick, downward pointing arrow, the load is applied onto the finite element model FE in a caudo-cranial direction C-C, for example on the uppermost vertebra 200n. In other words, the load is applied as if pulling the vertebra 200n down with respect to the reinforcement structure 30 attached to the spine 202 using pedicle screws 20₁₋ₘ.

In order to calculate the loading factor, a force at peak velocity, mean HU values, von Mises Stress Stress_{VonMises} values. The loading factor is calculated as the relationship between the local predicted stress weighted by the local bone quality using the formula:

$$\begin{matrix}Loading Factor [ \% ] = \frac{{Stress}_{von Mises}}{Failure Strength} \\ = mean ( \frac{{σ}_{von Mises , i}}{{σ}_{Fail , i}} )\end{matrix}$$

where σ_{vonMises,i} and σ_{Fail,i} are the resulting Stress_{VonMises} and the ultimate failure strength of element i in pedicle screws 200i, respectively.

For cases where a pair of pedicle screws is to be attached to the left and the right pedicle(s), a mean value between the left and the right pedicle is considered for determining the loading factor. This way, for every assessed parameter there was one data point per vertebra 200₁₋ₙ.

FIG. 6 shows a partial visual representation of the finite element model FE according to the present invention, illustrating the calculated load on a single pedicle screw 20₁ implanted into a vertebra 200₁.

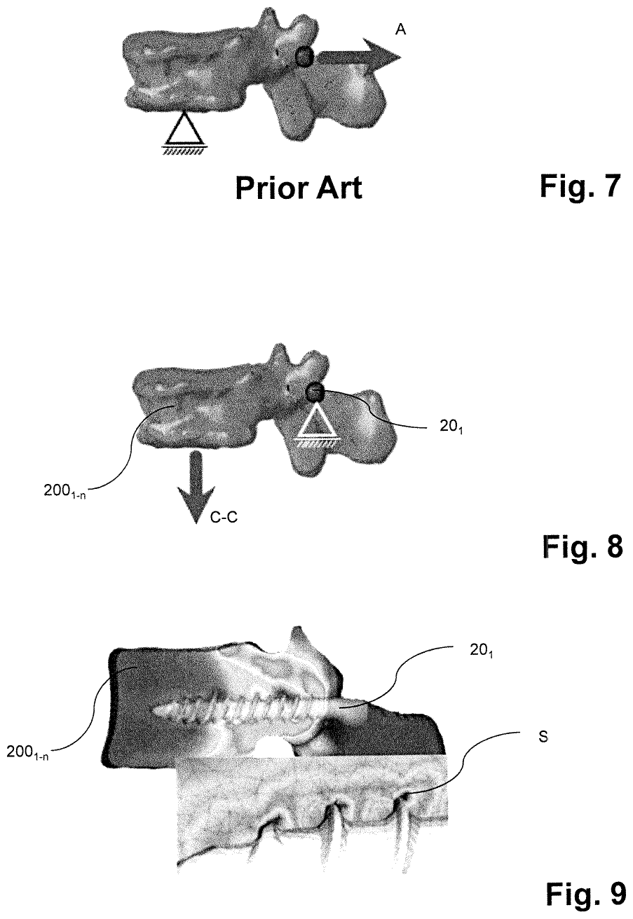

FIG. 7 shows visual representation of a finite element model according to the prior art for calculating a pull-out strength of a single pedicle screw for assessing a pedicle screw. However, it was recognized that prior art finite element models wherein load is applied onto the pedicle screws in an axial direction (see bold arrow on FIG. 7) do not yield meaningful assessment of the risk of pedicle screw loosening in patients.

As illustrated on FIG. 8, in contrast to the finite element model according to the prior art shown on FIG. 7, the finite element model FE according to the present invention is loaded in a caudo-cranial load. Loading the finite element model FE, according to the present invention, in a caudo-cranial direction C-C is advantageous as it allows a much more relevant, real-life assessment of the behavior of the pedicle screws 20₁₋ₘ in a patient, wherein the most significant stress applied onto the vertebrae 200₁₋ₙ is the gravitational force in an erect posture of the spine. The vertical gravitational force most commonly translates to a stress onto the pedicle screws 20₁₋ₘ which significantly deviates from an axial direction (with respect to a longitudinal axis of the pedicle screws), due to anatomical constraints on the orientation of the pedicle screws implanted into the vertebrae.

FIG. 9 shows a visual representation of the finite element model FE according to the present invention, illustrating the local predicted stress S in the bone structure of a pedicle 200₁ around a pedicle screw 20₁. As illustrated on the magnified view on the bottom right side of FIG. 9, significant stresses commonly occur around the thread of the pedicle screw 20₁.

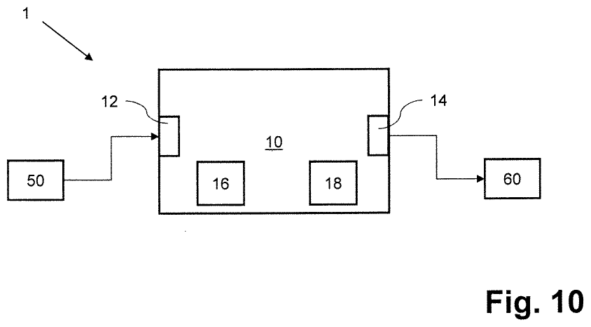

FIG. 10 shows a highly schematic block diagram of a system 1 for supporting selection between different types of pedicle screws 20₁₋ₘ according to the present invention. The system 1 comprises an imaging device 50 and a computing device 10 comprising a processing unit 16 and a memory unit 18 comprising instructions, which, when executed by the processing unit 16, cause the computing device 10 to carry out the method according to one of the embodiment disclosed herein. The processing unit 16 comprises any one or a combination of: a central processing unit CPU, a remote computing service, such as a cloud based software as a service, and/or a dedicated hardware circuitry, such as an ASIC or FPGA. The memory unit 14 comprises any one or a combination of: a volatile or non-volatile, optic, magnetic or semiconductor based data storage device, and/or a cloud based datastore.

The imaging device 50 may be a CT imaging device configured to capture preoperative CT image(s) of at least part of the spine 202 capturing at least two vertebrae 200₁₋ₙ of the spine 202, the imaging device 50 being communicatively connected to the computing device 10 via a data input interface 12. The data input interface 12 is any one of or a combination of a wired (e.g. Ethernet, DVI, HDMI, VGA) and/or wireless data communication interface (e.g. 4G, 5G, Wifi, Bluetooth, UWB).

The system 1 further comprises an output device 60, such as a computer screen, an augmented reality headset, or any device suitable to display a visual representation of the assessment of the plurality of different types of pedicle screws 20₁₋ₘ, the output device 60 being communicatively connected to the computing device 10 via a data output interface 14. The data output interface 14 is any one of or a combination of a wired (e.g. Ethernet, DVI, HDMI, VGA) and/or wireless data communication interface (e.g. 4G, 5G, Wifi, Bluetooth, UWB) is configured to transmit at least part of the assessment of the plurality of different types of pedicle screws 20₁₋ₘ, respectively a visual representation of the assessment.

1. A computer implemented method to support selection between different types of pedicle screws (201-m) to be applied in a spine (202) of a specific patient comprising the following method steps carried out by a computing device (10):
a. generating a spinal 3D-model (MSpine) of at least part of a spine (202) of the specific patient representing at least two vertebrae (2001-n) of the spine (202);
b. generating a reinforcement 3D-model (MRod) of a reinforcement structure (30) for interconnecting at least two vertebrae (2001-n) of the spine (202);
c. generating a finite element model (FE) corresponding to the spinal 3D-model (MSpine) and the reinforcement 3D-model (MRod), the finite element model (FE) representing:
i. the at least two vertebrae (2001-n); and
ii. the reinforcement structure (30) attached to the vertebrae (2001-n) using at least one pedicle screw (201-m)—of a first type of the different types of pedicle screws (201-m)—per interconnected vertebrae (2001-n), the reinforcement structure (30) forming an alternative load path outside the inter-connected vertebrae (2001-n);
d. applying a load onto the finite element model (FE) and determining loading factor(s) of at least one of the pedicle screws (201-m) using the finite element model (FE); and
e. outputting an assessment of the plurality of different types of pedicle screws (201-m) based on the calculated loading factor(s).

2. The computer implemented method according to claim 1, wherein the load is applied onto the finite element model (FE) in a caudo-cranial direction (C-C).

3. The computer implemented method according to claim 1, further comprising generating the spinal 3D-model (MSpine) using a database of
information of 3D-models of spines of people other than the specific patient.

4. The computer implemented method according to claim 1, further comprising:
a. receiving preoperative image(s); and
b. generating the spinal 3D-model (MSpine) further based on the pre-operative image(s).

5. The computer implemented method according to claim 4, further comprising:
a. extracting material properties indicative of a bone quality of the vertebrae (2001-n) from the preoperative image(s); and
b. assigning the extracted material properties to volume elements of the finite element model (FE).

6. The computer implemented method according to claim 5, wherein the loading factor of at least one of the pedicle screws (201-m) is determined as a relationship between a locally predicted stress and the bone quality of the corresponding vertebrae (2001-n).

7. The computer implemented method according to claim 1, wherein, if present, the spinal 3D-model (MSpine) and the finite element model (FE) comprise intervertebral disc(s) and/or fusion cage(s) arranged between the at least two vertebrae (2001-n).

8. The computer implemented method according to claim 1, further comprising:
a. creating at least a partial patient specific musculoskeletal model (MMuse); and
b. generating the finite element model (FE) further corresponding to the patient specific musculoskeletal model (MMuse).

9. The computer implemented method according to claim 1, wherein the output of the finite element model (FE) is recalculated based on at least one pedicle screw (201-m) positioned differently in the finite element model (FE).

10. The computer implemented method according to claim 1, wherein the output of the finite element model (FE) is recalculated based on at least one pedicle screw (201-m) of a second type of the different types of pedicle screws (201-m).

11. The computer implemented method according to claim 1, wherein assessment of the plurality of different types of pedicle screws (201-m) comprises outputting a warning signal with respect to each pedicle screw (201-m) with a load factor exceeding a critical load factor threshold.

12. A computing device (10) comprising a processing unit (16) and a memory unit (18) comprising instructions, which, when executed by the processing unit (16) cause the computing device (10) to carry out the method according to claim 1.

13. A system for supporting selection between different types of pedicle screws (201-m) to be applied in a spine (202) fixation of a specific patient, the system comprising:
a. an imaging device (50), in particular a CT imaging device, communicatively connected to the computing device (10), the imaging device (50) being configured to capture preoperative image(s) of at least part of the spine (202) capturing at least two vertebrae (2001-n) of the spine (202); and
b. a computing device (10) according to claim 12.

14. The system according to claim 13, further comprising an output device (60), such as a display device, for outputting the assessment of the plurality of different types of pedicle screws (201-m), the output device (60) being communicatively connected to the computing device (10).

15. A computer program product, comprising instructions, which, when carried out by a processing unit (16) of a computing device (10), cause the computing device (10) to carry out the method according to claim 1.
